(19) United States
(12) Patent Application Publication
(10) Pub. No.: US 2002/0188310 A1
(43) Pub. Date: Dec. 12, 2002

(54) Microfabricated surgical device

(76) Inventors: Kirk Partick Seward, Dublin, CA (US); Albert P. Pisano, Danville, CA (US); Philip Anthony Stupar, Oxnard, CA (US)

Correspondence Address: FISH & RICHARDSON P.C., 500 ARGUELLO STREET, SUITE 500, REDWOOD CITY, CA, 94063, US

(21) Appl. No.: 09/877,653

(22) Filed: Jun. 8, 2001

(51) Int. Cl. A61B 17/34; B29C 33/40; B29C 33/76

(52) U.S. Cl. 606/185; 604/272; 264/81; 264/219; 264/221; 264/317

(57) ABSTRACT

This invention relates to microfabricated surgical devices made of a conformally coated polymer.

### BACKGROUND

The present relates generally to surgical devices, and more particularly to microfabricated surgical devices and methods of making the same.

With the development of micro-fluidic systems on a chip comes the need for these chips to interact with the outside world. Microfabricated surgical devices, such as microneedles, are one such way to introduce samples to and extract solutions from organic tissue. However, current silicon and polysilicon microneedles fracture easily, and therefore must have their strength and toughness increased in order to be truly effective fluidic interconnects.

Out-of-plane, single crystal silicon microneedles can be made very sharp, but are limited in length by the thickness of the wafer from which they are made, and are somewhat fragile because the tips must be made hollow to facilitate fluid transport. In-plane single crystal silicon needles use deposited films to cap the fluid channel, and therefore have thin top wall thicknesses that can fracture under bending loads. Polysilicon microneedles use a deposited film for the entire structural layer and therefore are also likely to fracture under relatively small loads.

Although such previously fabricated microneedles have been proven to be effective fluidic interconnects, they have not been integrated into commercial devices because of the lack of strength and toughness. In addition, their brittle nature makes them hazardous to patients.

Silicon microneedles, for instance, will fracture before undergoing any plastic deformation. Such failure can be catastrophic. This type of failure is particularly hazardous for a microneedle application because this sort of rupture can lead to leakage of chemicals into the body that can be lethal in large dosages. Additionally, leaving behind particles of silicon in the body can have very perilous effects.

### SUMMARY

In one aspect, the invention features a microfabricated surgical device comprising an end portion and a body portion made of a conformally coated polymer.

In another aspect, the invention is directed to a microfabricated surgical device comprising a tip and a shaft made of conformal layer of polymer, wherein at least a portion of the shaft is hollow.

Various implementations of the invention may include one or more of the following features. The polymer may be Parylene. The polymer may be deposited by gas vapor deposition. The polymer may be selected from the group consisting of Parylene N, Parylene C, Parylene D, polystyrene, or Teflon®. The end portion of the device may include a metallic outer surface. The metallic outer surface may be made of a material selected from the group consisting of aluminum, gold, nickel, tungsten, zirconium, palladium, platinum, titanium, or alloys thereof. The end portion or the body portion of the device may include a reinforced section. A catheter may be joined to the device opposite the end portion.

In another aspect, the invention is directed to a microfabricated needle comprising a tip and a shaft each including a conformal polymer layer.

Various implementations of the invention may include one or more of the following features. The polymer may be selected from the group consisting of Parylene N, Parylene C, Paralene D, polystyrene, or Teflon®. The tip may include a metallic outer surface. The tip or shaft may include a reinforced section. A channel may be formed through at least a portion of the shaft, with a fluid entry port formed at a first end of the channel and a fluid exit port formed at a second end of the channel. The first end of the channel may be in fluid communication with a catheter. An interior cross-sectional dimension of the shaft may be between about 10 and 100 microns, while an exterior cross-sectional dimension of the shaft may be between about 50 and 250 microns. The device may have a length of between about 250 microns and five millimeters.

In another aspect, the invention is directed to a method of making a microfabricated surgical device. The method includes defining features of the device in a surface of a first substrate; joining a second substrate to the surface of the first substrate to define a mold cavity; conformally depositing a polymer in the mold cavity to form the device; and removing the device from the mold cavity.

In yet another aspect, the invention is directed to a method of making a microfabricated surgical device comprising: defining features of the device in a surface of a first substrate; forming a sacrificial release layer on the surface of the first substrate; joining a second substrate to the first substrate to define a mold cavity; forming a conformal layer of a polymer in the mold cavity; and removing the sacrificial release layer to release the device from the mold cavity.

Various implementations of the invention may include one or more of the following features. The first and second substrates may each be made of a material selected from the group consisting of silicon, glass or a polymer. The polymer may be either Parylene, polystyrene or Teflon®. The polymer may be deposited by gas vapor deposition. The features of a plurality of devices may be formed in the surface of the first substrate. The sacrificial release layer may be either an electroplated photoresist, a polymer, a metal, a semiconductor material, an oxide, or a microsoap.

In still another aspect, the invention features a method of making a microfabricated surgical device. The method comprises providing a substrate having a thickness approximately equal to a thickness of the device; defining features of the device by forming a mold from the substrate; forming a conformal layer of a polymer on the mold; and removing at least a portion of the mold such that the device includes a hollow portion.

In another aspect, the invention is directed to a method of making a microfabricated surgical device comprising: providing a substrate having a thickness approximately equal to a thickness of the device; defining features of the device by etching through the substrate to form a mold; forming a conformal layer of a polymer on the mold; and etching the mold such that the device includes a hollow portion.

Various implementations of the invention may include one or more of the following features. The mold can be etched such that the device includes a hollow shaft and a tip portion including the substrate material. Alternatively, the mold is etched such that the device has a hollow base, and shaft and tip portions including the substrate material. The substrate may be selected from the group consisting of silicon, metal, glass or a polymer. The conformal layer can be formed by gas vapor deposition of Parylene.

In still another aspect, the invention features a process for making a microneedle. The process includes defining features of the microneedle in a first surface of a first substrate; coating the surface of the first substrate with a first sacrificial layer; forming a metallic layer on the first sacrificial layer; coating the metallic layer with a second sacrificial layer and patterning the second sacrificial layer; joining a second substrate to the first substrate to define a mold cavity; conformally depositing a polymer layer in the mold cavity to form the microneedle; and etching the first and second sacrificial layers to remove the microneedle from the mold.

Various implementations of the invention may include one or more of the following features. The first and second substrates can each be made of a material selected from the group consisting of silicon, glass or a polymer. The polymer may be either Parylene, polystyrene or Teflon®. The polymer can be deposited by gas vapor deposition. The features of a plurality microneedles can be formed in the surface of the first substrate. The metallic layer may be formed by sputtering. The metal for the metallic layer can be selected from the group consisting of aluminum, gold, nickel, tungsten, zirconium, palladium, platinum, titanium, or alloys thereof. The first and second sacrificial layers can be an electroplated photoresist. The second sacrificial layer can be patterned such that the metallic layer, after the etching step, will remain only at a tip portion of the microneedle.

In still another aspect, the invention is directed a method of making a microfabricated surgical device comprising: defining features of the device in a surface of a first substrate; forming a sacrificial release layer on the surface of the first substrate; depositing a silicon nitride layer on the sacrificial release layer; joining a second substrate to the first substrate to define a mold cavity; forming a conformal layer of a polymer in the mold cavity; and removing the sacrificial release layer to release the device form the mold cavity.

An advantage of the invention is that it provides a microfabricated needle that is compliant enough to deflect with tissue motion. This needle can endure very large deflections, greater than 180° bends, without fracturing. The microfabricated needles are made of a conformally deposited polymer material, providing structures that can have wall thicknesses from less than one micron (μm) to more than 100 μm. This provides greater yields in manufacturing, fewer failures in the field, and less expensive packaging solutions for shipment. The deposition of a conformal polymer layer also permits formation of precise geometric features.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1I are schematic, cross-sectional views illustrating steps in the fabrication of a microfabricated needle. Cross-section A-A is across a reinforced section of the needle, for instance, the tip, while cross-section B-B is farther up the needle, along the needle shaft.

FIG. 2 is a schematic, perspective view of a mold used to make a microfabricated needle.

FIG. 3 is a schematic, perspective view illustrating a microfabricated needle having a metallic tip and edge penetration.

FIGS. 4A-4D are schematic, cross-sectional views illustrating steps in an alternative process for making a microfabricated needle. Cross-section A-A is across the needle shaft, while cross-section B-B is across a reinforced portion of the needle.

FIG. 5 is a schematic view illustrating a number of microfabricated needles that can be made using the process of FIGS. 4A-4D.

FIG. 6 is a schematic view illustrating a microfabricated needle having a reinforced tip and a hollow shaft.

FIG. 7 is a schematic view illustrating a microfabricated needle having a hollow base, and a reinforced shaft and tip.

FIG. 8 is a schematic, perspective view illustrating a microfabricated needle having a metallic tip and point penetration.

FIGS. 9A and 9B are schematic, perspective views illustrating mask processes for forming non-vertical walls in a substrate.

Like reference symbols and reference numbers in the various drawings indicate like elements.

### DETAILED DESCRIPTION

The present invention is directed to microfabricated surgical devices and methods of making the same. The present invention will be described in terms of several representative embodiments and processes in fabricating a microfabricated needle or microneedle. The described process may be used to make other microfabricated surgical devices, such as neural probes, lancets, in-vivo biological assay systems, cutting microtools, or devices including microtubing and incorporating, for example, channels and mixers.

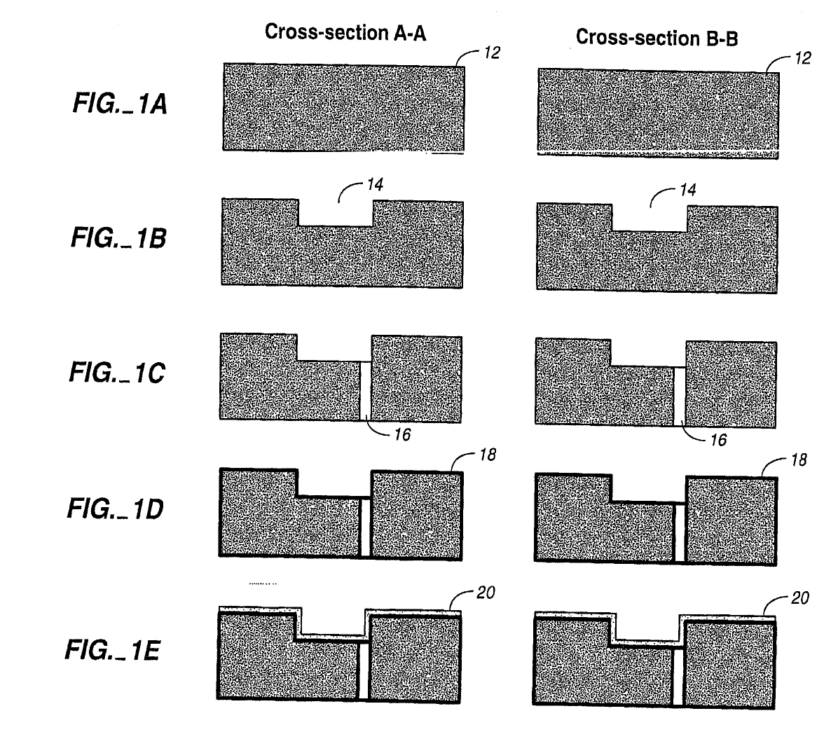

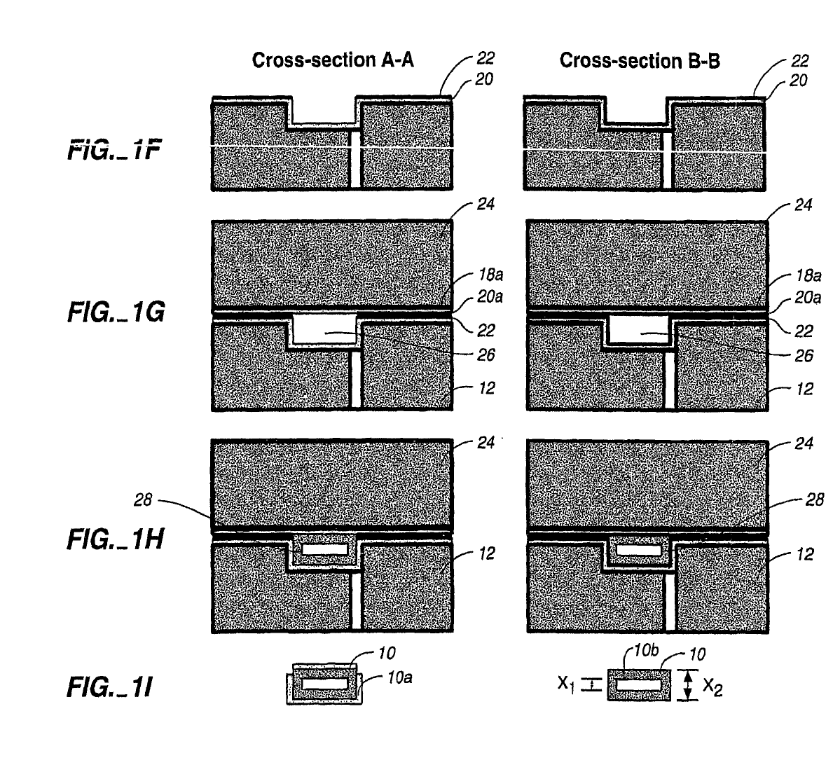

As shown in FIG. 1A, the fabrication of a microfabricated surgical device, such as a microfabricated needle or microneedle 10 (see FIG. 3), may start with a substrate such as a <100> single crystal silicon wafer 12 that is about 200 to 500 microns (μm) thick.

The wafer surface is subjected to a deep reactive ion etch (DRIE) to form a trench 14 having vertical sidewalls (FIG. 1B). The trench defines the features of the microneedle. The trench 14 may have a depth of between about 20 and 300 μm, and a length of between about 250 μm and five millimeters (mm).

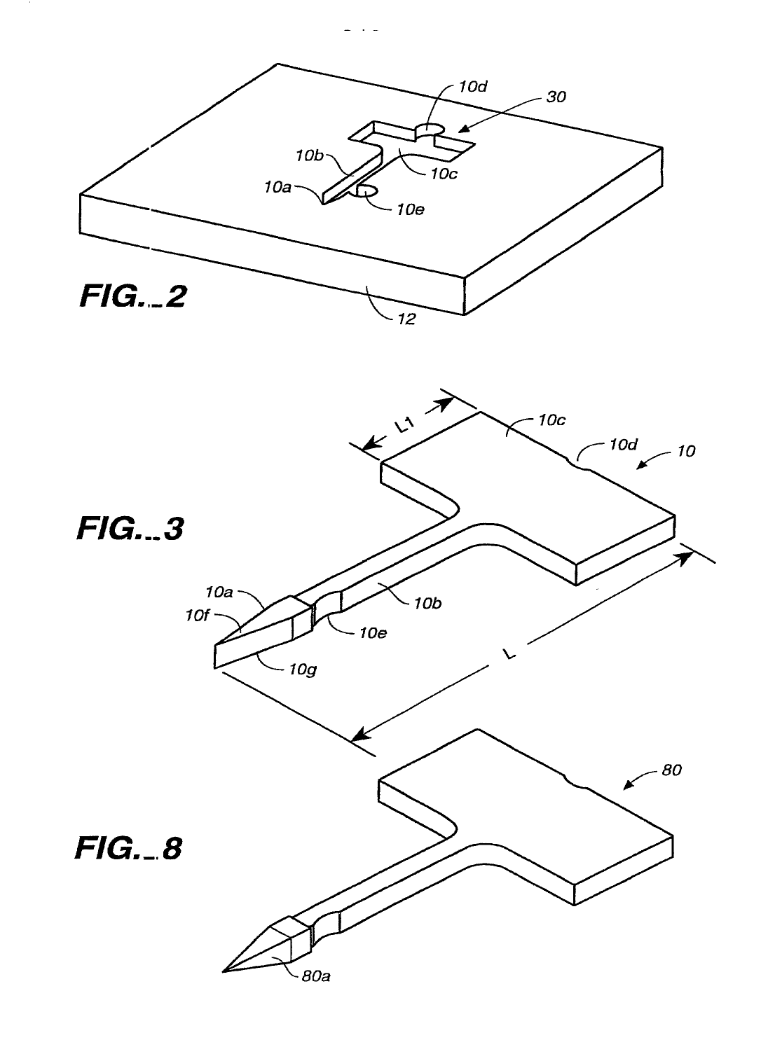

As shown in FIG. 2, the needle features may include a needle tip 10a, a needle shaft 10b, a needle base 10c, and needle entry and exit ports 10d and 10e, respectively. The inlet and outlet ports, alternatively, may be omitted or patterned with a different geometry at this stage of the process. Instead, after the microneedle structure is released from the mold, as discussed below, the ports can be selectively etched in the structure using an excimer or oxygen plasma laser.

Next, the wafer is subjected to a backside DRIE, as shown in FIG. 1C, to provide a channel or passageway 16 through which a polymer vapor, as explained below, is introduced into a mold cavity. Techniques other than DRIE, such as plasma etching or wet etching, may be used to form the trench and channel. The backside channel can be omitted and replaced by a frontside access port defined during the step of FIG. 1B.

A first, sacrificial release layer 18 is then formed on the wafer's surface (FIG. 1D). The sacrificial layer 18 can be formed by coating the wafer with an electroplated photoresist (EPRR). The sacrificial layer can also be formed by a thin coating of LPCVD polysilicon. The thickness of the layer 18 is substantially constant across the surface of the wafer and in the channel 16. Suitable photoresist materials are available from a number of suppliers including Shipley Microelectronics, Inc. of Marlborough, Mass. This layer may be between about one and ten μm thick.

The next step (FIG. 1E) is to form a metallic layer 20 on the side of the wafer including the microneedle features 14. The purpose of this optional step is to provide the needle tip or shaft (see FIGS. 2 and 3) with a reinforced metallic section that is sharp and more rigid than other portions of the needle 10. The layer 20 can be formed by sputter depositing a metal, such as aluminum, gold, nickel, tungsten, zirconium, palladium, platinum, titanium, or alloys of these metals, on the wafer. The layer 20 should be thick enough so that it is not porous; that is, it forms a contiguous film. The metal of layer 20, ideally, should not diffuse into the substrate material. Aluminum and certain aluminum alloys will diffuse into silicon. Thus, if such metals are used, an additional barrier layer (not shown) will be required between layer 20 and the wafer. This, obviously, complicates the process.

A second, sacrificial release layer 22 (FIG. 1F) is then formed on the metallic layer 20. The layer 22 may be an EPPR or polysilicon layer that is between about one and ten μm thick. The thickness of layer 22 is also substantially constant across the surface of the wafer. The layer 22, however, does not need to be formed in the channel 16. The layer 22 is patterned using, for example, photoresist (PR) lithography, to define where the polymer to be deposited, as discussed below, will be allowed to adhere to the metal layer 20 and where it will adhere to the second sacrificial layer 22.

As shown in FIG. 1G, a cap wafer or substrate 24 is then joined to the wafer 12 to form a three-dimensional mold cavity 26. The wafer 24 may also be a <100> single crystal silicon wafer, or it could be a glass or polymer wafer. The cap wafer has exposed metallic features like those that have been formed on the wafer 12. Thus, the cap wafer includes a sacrificial layer 18a and a metallic layer 20a like sacrificial layer 18 and metallic layer 20, respectively, of the wafer 12.

The wafers 12 and 24 may be bonded together. This bond may be performed in two steps. First a pre-bond is performed in which the two wafers are brought into close proximity allowing Van Der Wall forces to temporarily hold the wafers together. This pre-bond is performed with two clean, hydrophobic bare silicon surfaces. Wafers that are not particle free will have small voids that will lead to incomplete bonding. The pre-bonded wafers are then annealed at about 1000° C. for about one hour to allow the diffusion between the two wafers to permanently bond them together. The wafers may also be adhered together by the curing of thermoset photoresists.

The next step in the process is to perform a conformal polymer disposition (FIG. 1H). A Parylene C polymer may be gas vapor deposited into the mold cavity. Parylene is the generic name for the polymer poly-para-xylylene. Parylene C is the same monomer modified by the substitution of a chlorine atom for one of the aromatic hydrogens. Parylene C was chosen because of its conformality during deposition and its relatively high deposition rate, around 5 μm per hour.

The Parylene process is a conformal vapor deposition in which the substrate is kept at room temperature. A solid dimmer is first vaporized at about 150° C. and then cleaved into a monomer at about 650° C. This vaporized monomer is then brought into a room temperature deposition chamber, such as one available from Specialty Coating Systems of Indianapolis, Ind., where it absorbs and polymerizes onto the substrates and in the mold cavity. Because the mean free path of the monomer gas molecules is on the order of 0.1 centimeter (cm), the Parylene deposition is very conformal. The Parylene coating 28 is pin hole free at below a 25 nanometer (nm) thickness.

Due to the extreme conformality of the deposition process, Parylene will coat both the inside of the mold cavity 26, and the outside of the wafers 12 and 24. The Parylene coating 28 inside the mold cavity may be on the order of 20 to 80 μm thick, and more typically about 20 μm thick.

Other Parylenes, such as Types N and D, may be used in place of Parylene C. Also, other polymers, such as Teflon® or polystyrene, can be used. The important thing is that the polymer be conformally deposited. That is, the deposited polymer has a substantially constant thickness regardless of surface topologies or geometries.

Additionally, a fluid flood and air purge process could be used to form a conformal polymer layer in the mold cavity. Polymers that may be used in this process include polyurethane, an epoxy or a photoresist. The photoresist layers 18, 18a and 22 are next dissolved away from the structure. The photoresist layers may be etched away by an acetone, some other organic solvent, or a photoresist stripper. These materials destroy the photoresist layers, while not affecting the polymer or metal. The microneedle structure is released from the mold as the wafers separate in the etchant bath. The metal is removed from the microneedle where photoresist was present between the metal and polymer. The metal remains at the needle tip or the needle shaft for reinforcement (see FIG. 1I).

As can be seen from FIG. 3, the resultant microneedle 10 generally has a body portion and an end portion. More specifically, the microneedle includes a metallic tip 10a, and a polymer shaft 10b and base 10c. The needle tip 10a or termination point provides an insertion or penetration edge wherein a top surface 10f of the needle tip is a projection of its bottom surface 10g. The shaft and a channel through the base are hollow, permitting the injection of a fluid, for instance, into a patient via the inlet and outlet ports 10d and 10e, respectively.

The base 10c provides a mechanism for handling or assembly of the microneedle. The base, however, may be eliminated, if, for instance, the needle is to be placed at the tip of a catheter for use in interventional procedures. A catheter tip can be lined-up with the needle shaft end in the mold cavity, and as the polymer grows to create the needle structure, it encapsulates the catheter tip, fixing the needle in place.

The process involves the micromachining of a mold structure 30 from a substrate 12 (see FIG. 2). As discussed, the substrate may be silicon. It, however, could also be a glass or polymer material. Several thousand molds can be fabricated, for example, on a four-inch diameter wafer, leading to device batch fabrication.

By way of example, as shown in FIGS. 1I and 3, an individual microneedle may have an overall length L between about 250 μm and 5 mm. The length L₁ of the base portion, if present, may be between about 100 and 1,000 μm. The hollow, interior cross-sectional dimension x₁ of the shaft 10b may be on the order of 10 to 100 μm, while the shaft's exterior cross-sectional dimension x₂ is between about 50 and 250 μm.

Variations of the above-described process are possible. For instance, a “glass” encased polymer microneedle may be made by depositing a thin film of silicon dioxide, which is the sacrificial release layer, followed by a deposition of a thin film of silicon nitride. The mold is capped and the polymer is deposited, adhering to the silicon nitride. The silicon dioxide is removed in a hydrofluoric acid (HF) etch. The HF etch does not affect the polymer. The microneedle structure is released from the mold as silicon dioxide is dissolved. The resultant multi-layer structure has a polymer interior and a silicon nitride coating.

Other materials, such as tungsten carbide, silicon carbide or silicon dioxide, can be used instead of silicon nitride. Different sets of sacrificial layers or etchants may be required for such materials. The deposition of silicon nitride and these other materials may be accomplished using chemical vapor deposition (CVD) or low pressure chemical vapor deposition (LPCVD).

Such a microneedle is very rigid and sharp. This sort of process is feasible as the silicon nitride, the first deposited material, has a higher deposition temperature than the second deposited material, the polymer. As discussed, the polymer material is deposited at room temperature, while the silicon nitride is deposited at 835° C.

An alternative method for building a microneedle having a metallic tip or shaft reinforcement is to create the microneedle as in the process in FIGS. 1A-1I above, but without the metalization steps (FIGS. 1E, 1F). After releasing the microneedle from the mold, a thin metallic seed layer, such as titanium or one of other metals mentioned above, can then be sputtered onto the needle tip, and a subsequent electroplating step can be performed to grow metal on this seed layer. The thickness of this metal casing is tailored with the electroplating solution and the deposition voltage. The thickness, for instance, may be on the order of 1 to 30 μm.

This process could also be used to encase the microneedle shaft in a metallic layer. This metallic casing would add overall strength to the microneedle while maintaining its ductile framework.

Selective adhesion and release methods can also be used to cause metal adhesion or polymer release. Adhesion is aided by promoters like hexamethlydisilayane (HMDS) vapor, while release is enabled by a thin film of microsoap. The microsoap could be used in place of the sacrificial photoresist layers described above.

The microsoap is deposited in liquid form into the mold and then dried in heat or a vacuum. Patterning of the dried microsoap is performed by standard photolithography techniques and removal occurs in water or mild chemicals. The microsoap is patterned to provide adhesion or release in particular places in the mold. When the polymer is deposited onto this microsoap film, it will release in the water or mild chemicals. Thus, the microsoap provides a selective release or sacrificial layer.

A metallic layer, such as chromium, gold or titanium, could also be used as a selective release layer. Such a metallic layer can be sputtered deposited into the mold to a thickness of approximately two to five μm. The two wafers are then bonded together by, for example, solder bonding or by using a photoresist as a bond layer. A polymer is deposited into the mold cavity, and the metallic layers are subsequently selectively etched away by chemical etching, to release the device structure.

Thus, as described above, various release layers can be used. They include photoresists, oxides, metals and microsoaps. A polymer could also be used as a release layer, if it can be etched preferentially without affecting the polymer from which the device is fabricated. An example of such a polymer is SU-8 epoxy as available from Shipley Microelectronics, Inc.

Alternatively, instead of using a selective release layer, the device structure may be removed from the mold by mechanical ejection. Mechanical ejection can be performed by physically separating the two wafers and pulling the device structure away from the mold by a sprue or by injecting the device structure with an ejection pin through a hole in one of the wafers.

The microneedle structure discussed above was formed with vertical sidewalls produced by DRIE (see FIG. 1B). However, other sidewall geometries are possible, depending upon the etching technique used and the crystallographic microstructure of the single crystal silicon. Rounded features can be made in the plane of the wafer using isotropic wet chemical etching of silicon, and sloping sidewalls can be formed by anisotropic wet chemical etching. These sidewall geometries may be useful for different device configurations, for example, microneedles with filter plates or surgical devices that can cut sideways.

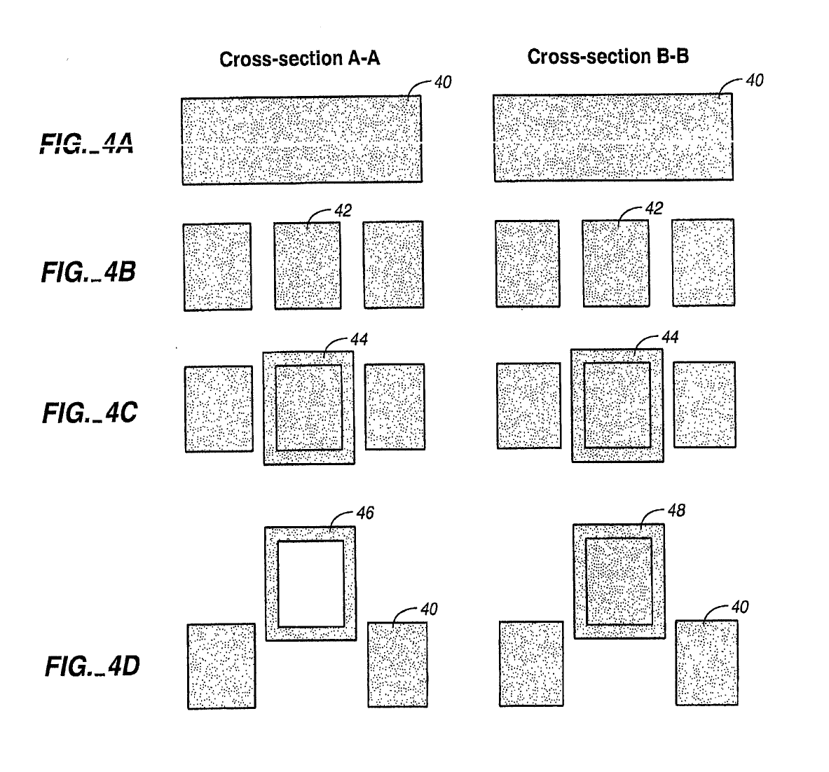

As shown in FIG. 4A, a sacrificial substrate process can be used to make a microfabricated device. This process can begin with a <100> single crystal silicon wafer or substrate 40 having a thickness equal to the desired thickness of the device. For instance, a wafer that is about 200 μm thick could be used. Also, other substrate materials, such as glass, a metal or a polymer, may be used.

A masking material (not shown) can be patterned onto the wafer to make inlet and outlet ports. The masking material may be a thick photoresist layer. Alternatively, the masking material may be left out all together, and the inlet and outlet ports can be etched into the structural layer material, discussed below, after its deposition.

The outline of the device 42, such as a microneedle, is then etched, for example, completely through the substrate using DRIE or STS deep silicon etching (FIG. 4B).

All four sides of the device outline are then coated with a conformal polymer structural layer 44, such as Parylene C (FIG. 4C). The Parylene C polymer deposits conformally at 5 μm per hour, thereby facilitating the deposition of a relatively thick structural layer. The thickness of layer 44 may be between about 1 and 50 μm.

The sacrificial silicon is now etched away to leave behind a hollow shaft 46 (FIG. 4D). The silicon etching can be done either in a heated potassium hydroxide (KOH) bath or in a xenon diflouride etcher. The xenon diflouride system has a maximum etch rate of around 10 μm per minute, and therefore takes approximately seven hours to completely undercut the shaft structure. Although KOH etches silicon at a much slower rate of 1 μm, it is the better method for etching Parylene because of the poor adhesion between the silicon and Parylene materials. This poor adhesion allows the liquid KOH to penetrate between the silicon and Parylene, and therefore etch away the sacrificial silicon much faster. The etch takes around eight hours to complete, giving an undercutting rate of 0.5 mm/hour. Although this is longer then the xenon diflouride etch time, it is in fact much faster overall because the xenon diflouride etcher has purge and cool down steps that triple the etch time. In addition, the wet KOH etch is preferable because of the ease of setup for long etch times.

If stiffer sections are required, the etch step can be stopped early so that the microneedle is not completely hollow. This technique can create a microneedle that has a hollow polymer shaft portion 46 and a tip portion 48 that is made-up of the substrate material, for example, silicon.

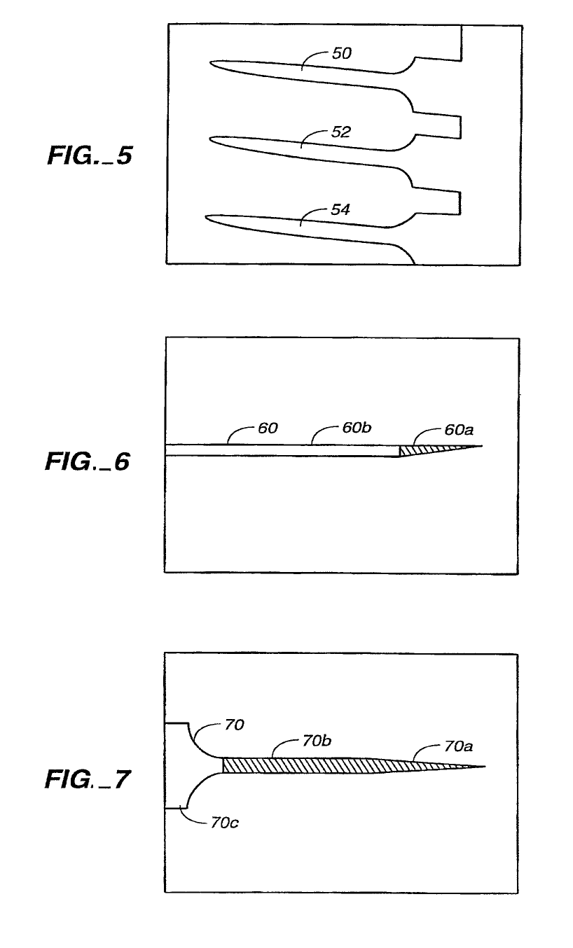

An array of microneedles 50, 52 and 54 made in accordance with the process of FIGS. 4A-4D is shown in FIG. 5. The entire sacrificial mold has been etched away to create completely hollow polymer needles.

The microneedles 60 and 70, however, illustrated in FIGS. 6 and 7, respectively, have different lengths of substrate material left behind to create stiffer or reinforced sections. The needle 60 has a hollow shaft 60a and a solid tip 60b. The needle 70 has a mostly solid shaft 70a and a solid tip 70b, with a needle base 70c being hollow. The stiffer sections increase the needle's buckling load as well as providing a shaper tip. The microneedles are thus strong enough to pierce very tough membranes.

A modification to the process outlined in FIGS. 1A-1I can be used to make a needle 80 wherein a needle tip 80a forms an insertion or penetration point (FIG. 8). The insertion point is advantageous as less force is necessary to break tissue than with an insertion edge microneedle (FIG. 3).

The process of FIGS. 1A-1I uses DRIE to produce deep trenches with vertical sidewalls. The mask used to protect the substrate in this etch setup is a thick layer (about 10 μm) of photoresist, for which the etching system has the selectively to etch the substrate at a much faster rate than the photoresist. However, the fact that the photoresist erodes during this etch leads to a process to create non-vertical sidewalls in the thick photoresist, leading to non-vertical sidewalls in the etched structure.

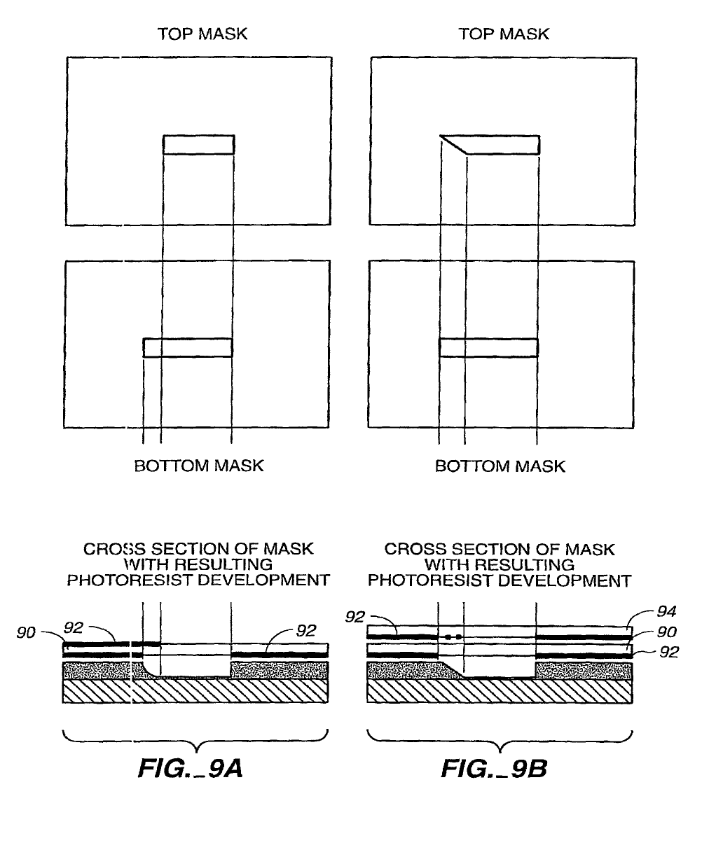

The two processes illustrated in FIGS. 9A and 9B show how the photoresist can be patterned to take advantage of erosion during etching. Conventional masking procedure uses contact lithography of a glass plate with chromium patterned on one side. Flood exposure with ultraviolet (UV) light breaks down the photoresist and subsequent chemical development removes the photoresist leaving behind the vertical sidewalls.

As shown in FIG. 9A, a glass mask plate 90 is patterned with chromium 92 on both sides so that the “contact” openings on the bottom side of the plate are larger than the “shadow” openings on the top side of the plate. This allows the UV light to pattern all features where both openings coincide, but only partially expose the openings covered by only the “shadow” mask. As seen, this would produce rounded sidewalls as the UV energy decreases with the distance it must travel beneath the “shadow” mask.

FIG. 9B illustrates a moving mask system, in which the contact mask 90 remains stationary on the wafer, defining where UV light is allowed to expose the photoresist, and a “shadow” mask 94 is translated across the opening (the mask moves in the plane of the page on which FIG. 9B appears and down across the opening of mask 90), allowing specified doses of UV light energy to the areas uncovered in the “shadow” mask. As seen, this produces tapered sidewalls with a geometry dictated by the speed of translation of the top mask and the mask opening of the top mask.

Microfabricated needles can be used to inject pharmaceutical agents into or extract biological samples from humans or animals while limiting injury or pain. The scale of these microneedles allows insertion into the human epidermis without penetrating deep enough for nerve reception. One application of this technology is insulin injection for diabetics who need a daily dosage of medication where pain and possible scarring occur with each conventional needle penetration.

These devices can also be used for interventional surgical methods in which a microneedle attached to the distal (inside the body) end of a catheter could penetrate an arterial wall with a microscale hole. Medical research has shown that damage to the inside of arteries caused by abrasion or lesion can seriously affect patients with sometimes drastic consequences such as vasospasm, leading to arterial collapse and loss of blood flow. Breach of the arterial wall through interventional surgical microneedles can prevent such problems.

The use of interventional surgical microneedles also allows highly localized pharmaceutical injections without the limitation of remaining external to the body. Common pharmaceutical procedures carried out with intravascular injections cause unnecessary flushing of the drugs throughout the body and filtering through the kidneys liver and the lymphatic system. On the other hand, localized injections allow slow, thorough integration of the drug into the tissue, thus performing the task more efficiently and effectively, saving time, money, drugs, and lives.

The microfabricated needle tip, for certain applications, can be coated with a blood-clotting agent such as heperin. These microneedles can also be used to introduce fluids to and extract fluids from a micro-fluidic system on a chip.

A number of embodiments of the invention have been described. Nevertheless, it will be understood that various modifications may be made without departing from the spirit and scope of the invention. Accordingly, other embodiments are within the scope of the following claims.

What is claimed is:

1. A microfabricated surgical device comprising: an end portion and a body portion made of a conformally coated polymer.

2. The microfabricated surgical device of claim 1 wherein the polymer is Parylene.

3. The microfabricated surgical device of claim 1 wherein the polymer is deposited by gas vapor deposition.

4. The microfabricated surgical device of claim 1 wherein the polymer is selected from the group consisting of Parylene N, Parylene C, Parylene D, polystyrene, or Teflon®.

5. The microfabricated surgical device of claim 1 wherein at least the end portion includes a metallic outer surface.

6. The microfabricated surgical device of claim 5 wherein the metallic outer surface is made of a metal selected for the group consisting of aluminum, gold, nickel, tungsten, zirconium, palladium, platinum, titanium, or alloys thereof.

7. The microfabricated surgical device of claim 1 wherein the end portion includes a reinforced section.

8. The microfabricated surgical device of claim 7 wherein the body portion includes a reinforced section.

9. The microfabricated device of claim 1 wherein a catheter is joined to the device opposite the end portion.

10. A microfabricated surgical device comprising:
a tip and a shaft made of a conformal layer of a polymer, wherein at least a portion of the shaft is hollow.

11. The microfabricated device of claim 10 wherein the polymer is Parylene.

12. The microfabricated device of claim 11 wherein the Parylene is deposited by gas vapor deposition.

13. The microfabricated device of claim 10 wherein the polymer is selected from the group consisting of Parylene N, Parylene C, Parylene D, polystyrene, or Teflon®.

14. The microfabricated device of claim 10 wherein at least the tip includes a metallic outer surface.

15. The microfabricated device of claim 14 wherein the metallic outer surface is made of a metal selected for the group consisting of aluminum, gold, nickel, tungsten, zirconium, palladium, platinum, titanium, or alloys thereof.

16. The microfabricated device of claim 10 wherein the tip includes a reinforced section.

17. The microfabricated device of claim 16 wherein the shaft includes a reinforced section.

18. The microfabricated device of claim 10 wherein a catheter is joined to the device opposite the tip.

19. The microfabricated device of claim 10 wherein an interior cross-sectional dimension of the shaft is between about 10 and 100 microns.

20. The microfabricated device of claim 10 wherein an exterior cross-sectional dimension of the shaft is between about 50 and 250 microns.

21. The microfabricated device of claim 10 having a length of between about 250 microns and five millimeters.

22. A microfabricated needle comprising a tip and a shaft each including a conformal polymer layer.

23. The microfabricated needle of claim 22 wherein the polymer is selected from the group consisting of Parylene N, Parylene C, Parylene D, polystyrene, or Teflon®.

24. The microfabricated needle of claim 22 wherein at least the tip includes a metallic outer surface.

25. The microfabricated needle of claim 22 wherein the tip includes a reinforced section.

26. The microfabricated needle of claim 25 wherein the shaft includes a reinforced section.

27. The microfabricated needle of claim 22 wherein a channel is formed through at least a portion of the shaft, and further including a fluid entry port formed at a first end of the channel and a fluid exit port formed at a second end of the channel.

28. The microfabricated needle of claim 27 wherein the first end of the channel is in fluid communication with a catheter.

29. The microfabricated needle of claim 22 wherein an interior cross-sectional dimension of the shaft is between about 10 to 100 microns, an exterior cross-sectional dimension of the shaft is between about 50 to 250 microns, and the microfabricated needle has a length of between about 250 microns and five millimeters.

30. A method of making a microfabricated surgical device comprising:
defining features of the device in a surface of a first substrate;
joining a second substrate to the surface of the first substrate to define a mold cavity;
conformally depositing a polymer in the mold cavity to form the device; and
removing the device from the mold cavity.

31. The method of claim 30 where the first and second substrates are each made of material selected from the group consisting of silicon, glass or a polymer.

32. The method of claim 30 wherein the polymer being deposited is either Parylene, polystyrene or Teflon®.

33. The method of claim 30 wherein the polymer is deposited by gas vapor deposition.

34. The method of claim 30 wherein the features of a plurality of devices are formed in the surface of the first substrate.

35. A method of making a microfabricated surgical device comprising:
defining features of the device in a surface of a first substrate;
forming a sacrificial release layer on the surface of the first substrate;
joining a second substrate to the first substrate to define a mold cavity;
forming a conformal layer of a polymer in the mold cavity; and
removing the sacrificial release layer to release the device form the mold cavity.

36. The method of claim 35 where the first and second substrates are each made of a material selected from the group consisting of silicon, glass or a polymer.

37. The method of claim 35 wherein the polymer is Parylene.

38. The method of claim 37 wherein the Parylene is deposited by gas vapor deposition.

39. The method of claim 35 wherein the sacrificial release layer is either an electroplated photoresist, a polymer, a metal, a semiconductor material, an oxide, or a microsoap.

40. A method of making a microfabricated surgical device comprising:
providing a substrate having a thickness approximately equal to a thickness of the device;
defining features of the device by forming a mold from the substrate;
forming a conformal layer of a polymer on the mold; and
removing at least a portion the mold such that the device includes a hollow portion.

41. A method for making a microfabricated surgical device comprising:
providing a substrate having a thickness approximately equal to a thickness of the device;
defining features of the device by etching through the substrate to form a mold;
forming a conformal layer of a polymer on the mold; and
etching the mold such that the device includes a hollow portion.

42. The method of claim 41 wherein the mold is etched such that the device includes a hollow shaft and a tip portion including the substrate material.

43. The method of claim 41 wherein the mold is etched such that the device has a hollow base, and shaft and tip portions including the substrate material.

44. The method of claim 41 wherein the substrate being provided is selected from the group consisting of silicon, metal, glass or a polymer.

45. The method of claim 41 wherein the conformal layer is formed by gas vapor deposition of Parylene.

46. A process for making a microneedle comprising:
defining features of the microneedle in a surface of a first substrate;
coating the surface of the first substrate with a first sacrificial layer;
forming a metallic layer on the first sacrificial layer;
coating the metallic layer with a second sacrificial layer and patterning the second sacrificial layer;
joining a second substrate to the first substrate to define a mold cavity;
conformally depositing a polymer layer in the mold cavity to form the microneedle; and
etching the first and second sacrificial layers to remove the microneedle from the mold.

47. The method of claim 46 where the first and second substrates are each made of a material selected from the group consisting of silicon, glass or a polymer.

48. The method of claim 46 wherein the polymer is either Parylene, polystyrene or Teflon®.

49. The method of claim 46 wherein the polymer is deposited by gas vapor deposition.

50. The method of claim 46 wherein the features of a plurality microneedles are formed in the surface of the first substrate.

51. The method of claim 46 wherein the metallic layer is formed by sputtering.

52. The process of claim 46 wherein the metal for the metallic layer being formed is selected from the group consisting of aluminum, gold, nickel, tungsten, zirconium, palladium, platinum, titanium, or alloys thereof.

53. The process of claim 46 wherein the first and second sacrificial layers being coated are each an electroplated photoresist.

54. The process of claim 46 wherein the second sacrificial layer is patterned such that the metallic layer, after the etching step, will remain only at a tip portion of the microneedle.

55. A method of making a microfabricated surgical device comprising:
defining features of the device in a surface of a first substrate;
forming a sacrificial release layer on the surface of the first substrate;
depositing a silicon nitride layer on the sacrificial release layer;
joining a second substrate to the first substrate to define a mold cavity;
forming a conformal layer of a polymer in the mold cavity; and
removing the sacrificial release layer to release the device form the mold cavity.
